(19) United States
(12) Patent Application Publication
(10) Pub. No.: US 2020/0121807 A1
(43) Pub. Date: Apr. 23, 2020

(54) Anti-Cancer Agents

(71) Applicant: UEA ENTERPRISES LIMITED, Norwich Norfolk (GB)

(72) Inventors: Heba ALSHAKER, Norwich Norfolk (GB); Qi WANG, Norwich Norfolk (GB); Shyam Srivats RASIPUR, Norwich Norfolk (GB); Dmitry PSHEZHETSKIY, Norwich Norfolk (GB)

(73) Assignee: UEA Enterprises Limited, Norwich Norfolk (GB)

(21) Appl. No.: 16/470,368

(22) PCT Filed: Dec. 19, 2017
(86) PCT No.: PCT/GB2017/053794

§ 371 Date: Jun. 17, 2019

(30) Foreign Application Priority Data
Dec. 20, 2016 (GB) 1621692.1

(51) Int. Cl. A61K 47/69; A61K 31/337; A61K 31/137; A61K 47/59; A61P 35/00

(52) U.S. Cl. CPC A61K 47/6911; A61K 31/337; A61P 35/00; A61K 47/593; A61K 31/137

(57) ABSTRACT

Provided is a pharmaceutical composition comprising a taxane and a sphingosine kinase 1 (SK1) inhibitor, a kit comprising a first pharmaceutical composition comprising a taxane and a second pharmaceutical composition comprising an SK1 inhibitor, and a taxane for use in a method of treating cancer, wherein the taxane is administered simultaneously, separately or sequentially with an SK1 inhibitor.

### FIELD OF THE INVENTION

The invention relates to methods of treating cancer and compositions therefor.

### BACKGROUND TO THE INVENTION

Taxanes (such as paclitaxel, cabazitaxel and docetaxel) are established anti-cancer agents; these are anti-mitotic agents, the principal mechanism of which is disruption of microtubule function. In addition, docetaxel induces apoptosis of cancer cells. Taxanes (especially docetaxel) are used in the treatment of breast, colorectal, lung, ovarian, prostate, liver, renal, gastric, head and neck cancers, and melanoma. However, docetaxel chemotherapy can unfortunately induce adverse effects such as non-hematologic toxicities and febrile neutropenia.

In the Western world, prostate cancer is now the most commonly diagnosed noncutaneous cancer in men. Taxane (e.g. docetaxel) chemotherapy is offered to patients relapsed on hormone therapy. This treatment improves life expectancy and overall life quality, but only extends survival for a median period of less than 3 months, thus any improvement in this response would be of clear benefit.

In the UK breast cancer is now the most commonly diagnosed noncutaneous cancer in women (55,000 new cases every year) and is the second leading cause of cancer-related death claiming approximately 12,000 lives every year. Paclitaxel chemotherapy is offered as part of both first and second line regimens for locally advanced or metastatic breast cancer, however it has been shown to provide only a small survival advantage. In this context, any incremental improvement in response to chemotherapy would be of clear benefit.

It is among the objects of this disclosure to address the aforementioned problems.

### SUMMARY OF THE INVENTION

Accordingly, provided is a pharmaceutical composition comprising a taxane and a sphingosine kinase 1 (SK1) inhibitor, preferably wherein the taxane is docetaxel and/or the SK1 inhibitor is fingolimod.

Preferably, the taxane and the SK1 inhibitor are both comprised within a nanoparticle.

In preferred embodiments, said nanoparticle is configured such that, in vivo, release of the SK1 inhibitor from the nanoparticle starts at or before release of the taxane from the nanoparticle and/or initial release of the SK1 inhibitor from the nanoparticle occurs at the same or a greater rate than initial release of the taxane from the nanoparticle, and/or each of the taxane and the SK1 inhibitor is encapsulated by and/or covalently linked to a biodegradable and biocompatible polymer within the nanoparticle (e.g. wherein the SK1 inhibitor is covalently bound to a biodegradable and biocompatible polymer via an ester linkage and wherein the taxane is bound to a biodegradable and biocompatible polymer via an amide linkage). In other preferred embodiments, the taxane is encapsulated by and/or covalently linked to a biodegradable and biocompatible polymer within the nanoparticle, said polymer is surrounded by a liposome shell, and the SK1 inhibitor is contained within the lipid bilayer of the liposome.

In alternative, preferred embodiments, the taxane is comprised within a first nanoparticle and the SK1 inhibitor is comprised within a second nanoparticle.

Preferably, said first and second nanoparticles are configured such that, in vivo, release of the SK1 inhibitor from the second nanoparticle starts at or before release of the taxane from the first nanoparticle and/or initial release of the SK1 inhibitor from the second nanoparticle occurs at the same or a greater rate than initial release of the taxane from the first nanoparticle, and/or each of the taxane and the SK1 inhibitor is encapsulated by and/or covalently linked to a biodegradable and biocompatible polymer within the first and second nanoparticles, respectively (e.g. wherein the SK1 inhibitor is covalently bound to a biodegradable and biocompatible polymer via an ester linkage and wherein the taxane is bound to a biodegradable and biocompatible polymer via an amide linkage).

Also provided is a kit comprising a first pharmaceutical composition comprising a taxane and a second pharmaceutical composition comprising an SK1 inhibitor. Preferably, in the first pharmaceutical composition, the taxane is comprised within a first nanoparticle and, in the second pharmaceutical composition, the SK1 inhibitor is comprised within a second nanoparticle (e.g. wherein the first and second nanoparticles are configured as described as above in relation to first and second nanoparticles).

Also provided is a taxane for use in a method of treating cancer, wherein the taxane is administered simultaneously, separately or sequentially with an SK1 inhibitor.

In preferred embodiments, in said method the taxane is administered after the SK1 inhibitor has been administered.

In other preferred embodiments, in said method the taxane is administered simultaneously with the SK1 inhibitor, e.g. by administration of a pharmaceutical composition according to any one of the aspects described above, co-administration of the pharmaceutical compositions as defined within the kit aspects above, or administration of a pharmaceutical composition formed by the combination of the pharmaceutical compositions as defined within the kit aspects above.

Preferably, said cancer is breast cancer or prostate cancer (e.g. castrate-resistant prostate cancer).

### BRIEF DESCRIPTION OF THE FIGURES

FIG. 1 shows the low efficacy of 5 mg/kg docetaxel against primary human prostate tumours established in nude mice, which is potentiated by addition of an SK1 inhibitor (SK1-II). BalbC/nude immune deficient mice were injected with 1×10⁶ PC-3 metastatic prostate cancer cells and tumours were grown for 3 weeks. Mice were randomized in groups (n=7) according to treatment as shown in figure, and treated for 2 weeks every second day. Tumour weight at week 5. Columns, mean of three experiments performed in triplicate. Bars, SE. * p<0.05 versus control.

FIG. 2 shows the increased efficacy of FTY720 and docetaxel combination in primary human breast tumours established in nude mice. BalbC/nude immune deficient mice were injected with 1×10⁶ MDA-MB-231 metastatic triple negative breast cancer cell and tumours were grown for 3 weeks. Mice were randomized in groups (n=7) according to treatment as shown in figure, and treated for 2 weeks every second day. Tumour weight at week 5. Columns, mean of three experiments performed in triplicate. Bars, SE. * p<0.05 versus control.

FIG. 3 shows a scheme of core shell lipid-polymer hybrid nanoparticle (CSLPHNP) synthesis and drug loading, using PEGylated lipids. DTX=docetaxel.

FIG. 4 shows characterisation of CSLPHNPs, including: SEM imaging of (A) PLGA cores and (B) CSLPHNPs (20,000× magnification; scale bar: 1 μm); (C) dynamic light scattering measurement shows the size distribution of PLGA cores (dash line on left) and CSLPHNPs (solid line on right); (D) temporal release of FTY720 (black squares) and docetaxel (white circles) at pH5 was measured by liquid chromatography-tandem mass spectrometry (LC-MS/MS). Points, mean of three experiments performed in triplicate. Bars, SE.

FIG. 5 shows in vitro assessment of CSLPHNPs. Hormone-refractory metastatic prostate cancer PC-3 cells were treated with free drugs or CSLPHNPs for 72 hours. (A) Fluorescent/phase contrast images of PC-3 cells after incubation with CSLPHNPs containing Rhodamine B. (B) CSLPHNPs uptake assessed by fluorescent microscopy (A) was quantified by ImageJ. (C) Cytotoxicity of CSLPHNPs was assessed using the MTT assay (24 hr: dark shading; 48 hr: light shading; 72 hr: hatching). (D) PC-3 cells were treated for 48 h as indicated in figure and SK1 activity was measured by radiolabelling. Columns, points, percentage relative to the control. Data is expressed as Mean±SE (n=3). * p<0.05 versus control, ** p<0.05 versus free drug treatment (DTX+FTY). (E) Cytotoxicity of free drugs was assessed using the MTT assay (48 hr: dark shading; 72 hr: light shading; 96 hr: no shading); the final two columns showing sequential administration of docetaxel on day 1 (D1) followed by FTY720 on day 2 (D2), or administration of FTY720 on day 1 (D1) followed by docetaxel on day 2 (D2).

FIG. 6 shows in vivo assessment of CSLPHNPs. NSG immune deficient mice were injected with 1×10⁶ PC-3 cells and tumours were grown for 3 weeks. Mice were randomized in groups (n=7) according to treatment as shown in figure, and treated for 2 weeks every second day. All nanoparticles were labelled with CF488 fluorophore. (A) Tumour weight at week five. (B) Intratumoural SK1 activity. (C) Fluorescent microscopy of mouse organs and primary tumours. Graph below indicates the levels of fluorescence in each organ quantified using ImageJ. (D) Relative body weight. (E) White cell count (WCC). Data is presented as Mean±SE, (n=7), *, p<0.05 versus control.

FIG. 7 shows physicochemical release kinetics of CSLPHNPs in neutral media. Temporal release of FTY720 (black squares) and docetaxel (white circles) at pH 7.4 was measured by liquid chromatography-tandem mass spectrometry (LC-MS/MS). Points, mean of three experiments performed in triplicate. Bars, SE.

FIG. 8 shows Long-term stability of the CSLPNPs. (A) Size and (B) polydispersity index (PDI) of CSLPNPs in PBS, DMEM, DMEM+FBS, FBS, and human plasma, which were monitored for a period of 5 days. (C) Size and PDI measurements of CSLPNPs suspended in PBS and stored at 4° C. for up to 30 days. Data is expressed as Mean±SE (n=3).

FIG. 9 shows particle size, polydispersity index and zeta potential of CSLPNPs.

FIG. 10 shows CSLPNPs uptake in DU145 cell line. Fluorescent/phase contrast images of hormone-refractory metastatic prostate cancer DU145 cells that were treated with CSLPHNPs containing Rhodamine B for 72 hours.

FIG. 11 shows cytotoxicity of CSLPNPs on DU145 cell line. Hormone-refractory metastatic prostate cancer DU145 cells were treated with free drugs or CSLPHNPs for 72 hours. Cytotoxicity of treatments was assessed using the MTT assay (24 hr: dark shading; 48 hr: light shading; 72 hr: hatching). Data is expressed as Mean±SE (n=4). * p<0.05 versus control.

FIG. 12 shows evaluation of potential side effects of various drug formulations. NSG immune deficient mice were injected with 1×10⁶ PC-3 cells and tumours were grown for 3 weeks. Mice were randomized in groups (n=7) according to treatment as shown in figure, and treated for 2 weeks every second day. At Day 30 mice were sacrificed and liver weight measurements were performed. Liver weight was normalised to control group (healthy group no treatment). *: p<0.05 vs. control group (ANOVA). Data is expressed as Mean±SE (n=7).

FIG. 13 shows nanoparticle (NP) characterization and drug release profile. PLGA NPs' representative high resolution images from scanning electron microscopy (A) and transmission electron microscopy (B). Scale bars indicate 1 μm and 100 nm respectively. (C) Distribution of hydrodynamic diameter of PLGA NPs measured using dynamic light scattering. (D) Physiochemical release kinetics shows sustained release of docetaxel (white circles) and FTY720 (black squares) quantified over a 120 hour time period. Points, mean of three independent experiments performed in triplicate; Bars, SE.

FIG. 14 shows NP in vitro studies. Uptake studies of PLGA NPs in PC3 cells—fluorescent live cell imaging; (A) control and (B) 48 hour exposure to PLGA NPs 100 ug/ml; (C) Data from Flow cytometry—up to 72 hour exposure to PLGA NPs, fluorescent signal doubled after 24 hour. Mean FL-2 was normalised to control. (D) MTT cell viability assay in two prostate (PC3 and DU145) and two breast (MDA-MB-231 and BT549) cancer cell lines. Within each cell line, the bars, left to right, are: control; free drug treatment of 5 nM docetaxel plus 5 μM FTY720; empty NPs; NP comprising 5 nM docetaxel plus 5 μM FTY720. Cells were treated with free drugs or PLGA NPs for 48 hours. Bars, SE. *: p<0.05 compared to control.

FIG. 15 shows that PLGA NPs demonstrate reduced toxicity in comparison with systemic therapy. (A) Liver weight was normalised to control group, which did not receive any treatment. (B) Spleen weight was normalised to control group, which did not receive any treatment. (C) Serum alanine transaminases (ALT) (a marker for liver dysfunction) was measured in treatment groups. (D-F) Effect of PLGA NPs and free drug treatment on blood count: (D) white cell count (WCC), (E) red blood cells (RBC) and (F) haemoglobin (Hb). *: p<0.05 vs. control group (ANOVA).

FIG. 16 shows a comparison of sequential versus simultaneous administration of docetaxel and FTY720 (each at 5 mg/kg), within nanoparticles, against primary human prostate tumours established in nude mice. BalbC/nude immune deficient mice were injected with 1×10⁶ PC-3 metastatic prostate cancer cells and tumours were grown for 3 weeks. Mice were randomized in groups (n=7) according to treatment as shown in figure. Tumour weight at week 5. (i) FTY720 nanoparticle on day 1, docetaxel nanoparticle on day 2. (ii) Combined docetaxel and FTY720 nanoparticle. (iii) Simultaneous administration of FTY720 and docetaxel nanoparticles (mixed). Columns, mean of three experiments performed in triplicate. Bars, SE. * p<0.05 versus control.

### DETAILED DESCRIPTION OF THE INVENTION

### General

Provided herein are certain pharmaceutical compositions, for use in certain medical indications and per se. ‘Pharmaceutical composition’ reflects e.g. the clinical setting in which these compositions are intended to be used.

Provided is a pharmaceutical composition comprising a taxane and a sphingosine kinase 1 (SK1) inhibitor. A taxane is a member of the class of diterpenes that feature a taxadiene core. Taxanes of particular interest include paclitaxel, cabazitaxel and (most preferably) docetaxel.

An SK1 inhibitor is any compound that inhibits the activity of the SK1 enzyme. The SK1 inhibitor is preferably selected from the group consisting of: dihydroshingospine (DHS), dimethylshingospine (DMS), F-12509a or B-5354C (Kono et al (2002). Inhibition of recombinant sphingosine kinases by novel inhibitors of microbial origin, F-12509A and B-5354c. The Journal of Antibiotics (Tokyo), 55, 99-103.), SK1-I, SK1-II, fingolimod, SKI-178, Compound V, (S)-FTY720 vinylphosphonate, Safingol, LCL 146, LCL 351, bag, 9ab and 12aa, as described in Alshaker et al (2013) Adv Cancer Res. 117:143-200, and others described in Lynch et al (2016) Expert Opin Ther Pat. December; 26(12):1409-1416.

The most preferred SK1 inhibitor is fingolimod, also know as FTY720 (sometimes shortened to FTY). This compound is a functional antagonist of sphingosine-1-phosphate receptors as well as an SK1 inhibitor, and is currently clinically used to treat multiple sclerosis by inducing lymphopenia and T-cell sequestration to lymph nodes (which is a key obstacle for fingolimod use in cancer patients).

In particularly preferred embodiments, the taxane is docetaxel and the SK1 inhibitor is fingolimod.

Pharmaceutically-acceptable salts of the taxane and of the SK1 inhibitor are explicitly contemplated. The composition may additionally comprise one or more pharmaceutically acceptable excipients or diluents.

The inventors have found (see Example 1) that, using mouse models of prostate and breast cancer, treatment using a combination of a taxane (docetaxel) and SK1 inhibitor (SK1-II or FTY720) had a much greater effect on tumour size compared with either active agent used alone, particularly in the former model, where the effect for the combination treatment was more than additive. This might pave the way for e.g. increased clinical efficacy or equivalent efficacy at lower doses (and hence potentially greater safety profile)—e.g. a taxane dose (within the combination therapy) one quarter of that required for effective treatment with taxane alone.

### ‘Combined’ Nanoparticles

In preferred embodiments, the taxane and the SK1 inhibitor are both comprised within a (combined) nanoparticle, to e.g. assist with co-delivery of the two active compounds and/or reduction of systemic absorption and hence adverse effects. By “comprised within a/the nanoparticle” or “within a/the nanoparticle” (here, and herein) we mean e.g. forming or forming part of a/the nanoparticle.

The nanoparticle is preferably substantially spherical, and/or preferably has a diameter (for example as measured by e.g. dynamic light scattering or zeta sizing) of 10 nm to 300 nm, preferably 20 nm to 300 nm, more preferably 20 nm to 200 nm. A diameter of 10 nm or more (preferably 20 nm or more) reduces or eliminates clearance of the nanoparticle by the kidneys.

In the composition of the invention, said nanoparticle might exist as a population of different sized nanoparticles, in which case (again, for example, as measured by e.g. dynamic light scattering or zeta sizing) it is preferred that the average particle size/diameter (by number) is between 10 nm and 300 nm, preferably between 20 nm and 300 nm, more preferably between 20 nm and 200 nm, more preferably between 50 nm and 175 nm. In addition, or alternatively, it is preferred that less than 10%, preferably less than 5%, preferably less than 1%, and preferably substantially none of the nanoparticles have a particle size/diameter of 10 nm or less, preferably 20 nm or less. In addition, or alternatively, it is preferred that at least 50%, preferably at least 75%, preferably at least 90%, preferably at least 95%, of the nanoparticles have a size/diameter of between 10 nm and 300 nm, preferably between 20 nm and 300 nm, more preferably between 20 nm and 200 nm, more preferably between 50 nm and 175 nm.

Preferably, for such nanoparticle embodiments, the pharmaceutical composition is an aqueous composition. In preferred embodiments, the composition has a pH of 7 or more, preferably of between 7 and 8.

Preferably, the nanoparticle is configured such that it (only) releases one or both active agents at a pH of less than 7, preferably at a pH of less than 6, preferably at a pH of 5.5 or less, preferably a pH of 5 or less, or that it releases one or both active agents at a greater rate at any such pH compared with a pH not above selected preferred range.

Preferably, the nanoparticle is configured such that, in vivo (and preferably once inside a cell, preferably a cancer cell), release of the SK1 inhibitor from the nanoparticle starts at or before release of the taxane from the nanoparticle and/or initial release of the SK1 inhibitor from the nanoparticle occurs at the same or a greater rate than initial release of the taxane from the nanoparticle.

In particular embodiments, each of the taxane and the SK1 inhibitor is encapsulated by and/or covalently linked to a biodegradable and biocompatible polymer within the nanoparticle. The two actives might or might not be encapsulated by or bound to the same type of polymer. Preferred embodiments include a) only the taxane being covalently bound to a polymer and b) both taxane/SK1 inhibitor being covalently bound to a polymer.

Covalent linkage between the taxane/SK1 inhibitor and the polymer is preferably by means of an ester bond or an amide bond.

Preferably, the SK1 inhibitor is covalently bound to a biodegradable and biocompatible polymer via an ester linkage and the taxane is bound to a biodegradable and biocompatible polymer via an amide linkage. In this way, one might promote the earlier and/or greater initial rate of release of the SK1 inhibitor compared with the taxane, given the greater propensity for cleavage of ester bonds in vivo compared with amide bonds.

In alternative embodiments, the taxane is encapsulated by and/or covalently linked to a biodegradable and biocompatible polymer within the nanoparticle, said polymer is surrounded by a liposome shell, and the SK1 inhibitor is contained within the lipid bilayer of the liposome. In this way, one might promote the earlier and/or greater initial rate of release of the SK1 inhibitor compared with the taxane, given the respective steric positions of the two actives. Again, any covalent linkage between the taxane and the polymer is preferably by means of an ester bond or an amide bond.

By biodegradable, we mean for example that the polymer is degraded in vivo to its base monomers. By biocompatible, we mean for example not having significant toxic or injurious effects on a biological system (e.g. a mammalian, particularly a human, body) (at least for a significant portion of a general or treated population) and/or having characteristics/effects recognised and established as suitable for use in vivo.

The term ‘polymer’ includes a copolymer.

Preferably, the or each biodegradable and biocompatible polymer comprises or consists of a polyester, preferably one comprising one or more of the following monomers: D,L-lactide, D-lactide, L-lactide, D,L-lactic acid, D-lactic acid, L-lactic acid, glycolide, and glycolic acid. Preferably therefore the or each biodegradable and biocompatible polymer comprises or consists of polylactic acid (PLA), polyglycolide (PGA) or, preferably, poly(lactic-co-glycolic acid) (PLGA).

When each of the taxane and the SK1 inhibitor is encapsulated by and/or covalently linked to a biodegradable and biocompatible polymer within the nanoparticle, and for example no outer shell (e.g. liposome) is present, the nanoparticle can be adapted/functionalised such that part or all of the outer surface of the nanoparticle comprises e.g. a carboxylic acid functional group (enabling well-established chemical reactions to be applied to attach different moieties e.g. targeting peptides to the surface of nanoparticles via amine-carboxylic acid conjugation), a glucosamine group (to aid targeting to cells [e.g. cancer cells] with high metabolic rate), and/or a polyethylene glycol (PEG) group (PEGylation acts e.g. to increase solubility, prolong plasma half-life and reduce immunogenicity.) Preferably, any such functionalising group is incorporated into the nanoparticle by covalently binding said group to a polymer therein.

Alternatively, an outer liposome shell can be provided, and this can be adapted/functionalised as described below. In this situation, the nanoparticle can be considered to comprise a polymer ‘core’ and a liposome shell.

This former construction is exemplified by the nanoparticles (NPs) described in Example 3 below, which consist of a complex of PLGA ester-linked to fingolimod, PLGA amide-linked to docetaxel, and PLGA amide-linked to glucosamine, where (sequential) release of fingolimod and docetaxel preferentially appears to occur at lysosomal pH, at least in a cell-free system.

The inventors have found (see Example 3) that, in vitro, these NPs showed equivalent or greater anti-cancer properties against prostate and breast cancer cell lines compared with the free drug combination. Using in vivo studies (breast cancer mouse model), it was shown that the NPs have comparable anti-cancer activity with the free drug combination, but display a better safety profile, especially in terms of body and spleen mass, and liver mass and function and, crucially, limited leukopenia/lymphopenia, together with limited or no adverse effect in terms of red blood cell count and haemoglobin levels.

When the taxane is encapsulated by and/or covalently linked to a biodegradable and biocompatible polymer within the nanoparticle, said polymer (or polymer ‘core’) is surrounded by a liposome shell, and the SK1 inhibitor is contained within the lipid bilayer of the liposome, the (e.g. solid) polymeric core acts as a scaffold to encapsulate the taxane and the lipid shell enveloping the core prevents internal drug leakage.

Preferably, the liposome is adapted/functionalised as described above. Preferably, this is achieved by covalent linkage of the functionalising group with a lipid(s) of the liposome's lipid bilayer.

This latter construction is exemplified by the core shell lipid-polymer hybrid nanoparticles (CSLPHNPs) described in Example 2 below, with PLGA core containing docetaxel surrounded by a liposome containing fingolimod, where (sequential) release of fingolimod and docetaxel preferentially occurs at lysosomal pH compared with plasma pH.

The inventors have found (see Example 2) that, in vitro, CSLPHNPs showed greater anti-cancer properties against prostate cancer cell lines compared with the free drug combination (which in turn showed greater efficacy compared with the each free drug on its own). Using in vivo studies (prostate cancer mouse model), it was shown that CSLPHNPs have comparable anti-cancer activity with the free drug combination, but display a better safety profile, especially in terms of body mass and liver mass and, crucially, absence of leukopenia/lymphopenia.

### ‘Singular’ Nanoparticles

In alternative embodiments, the taxane (preferably docetaxel) is comprised within a first nanoparticle and the SK1 inhibitor (preferably fingolimod) is comprised within a second nanoparticle. Each of these nanoparticles can be shaped or sized or have a size distribution or have a pH release profile as described above. The composition comprising the first and second nanoparticles is preferably an aqueous composition, preferably with a pH of 7 or more, preferably of between 7 and 8.

Preferably, said first and second nanoparticles are configured such that, in vivo (and preferably once inside a cell, preferably a cancer cell), release of the SK1 inhibitor from the second nanoparticle starts at or before release of the taxane from the first nanoparticle and/or initial release of the SK1 inhibitor from the second nanoparticle occurs at the same or a greater rate than initial release of the taxane from the first nanoparticle.

Preferably, each of the taxane and the SK1 inhibitor is encapsulated by and/or covalently linked to a biodegradable and biocompatible polymer within the first and second nanoparticles, respectively. The two actives might or might not be encapsulated by or bound to the same type of polymer. Preferred embodiments include a) only the taxane being covalently bound to a polymer and b) both taxane/SK1 inhibitor being covalently bound to a polymer. Covalent linkage is preferably by means of an ester bond or an amide bond. The biodegradable and biocompatible polymer(s) is as described above.

Preferably, the SK1 inhibitor is covalently bound to a biodegradable and biocompatible polymer via an ester linkage and the taxane is bound to a biodegradable and biocompatible polymer via an amide linkage. In this way, one might promote the earlier and/or greater initial rate of release of the SK1 inhibitor compared with the taxane, given the greater propensity for cleavage of ester bonds in vivo compared with amide bonds.

Note that each of the first and second nanoparticles (and/or any outer liposome shell thereof) can be adapted/functionalised as described above.

This arrangement of first and second nanoparticles is exemplified by the separate nanoparticles (NPs) described in Example 4 below, which consist of a) PLGA amide-linked to docetaxel and PLGA amide-linked to glucosamine and b) a complex of PLGA ester-linked to fingolimod and PLGA amide-linked to glucosamine. Therein the inventors have found that, in vivo (prostate cancer mouse model), simultaneous administration of fingolimod and docetaxel, in nanoparticles, either as a mix of separate NPs or using a combined NP, has a greater anti-cancer activity compared with administration of a fingolimod NP followed by a docetaxel NP.

### Kits

Also provided is a kit comprising a first pharmaceutical composition comprising a taxane (preferably docetaxel) and a second pharmaceutical composition comprising an SK1 inhibitor (preferably fingolimod).

Preferably, in the first pharmaceutical composition, the taxane is comprised within a first nanoparticle and, in the second pharmaceutical composition, the SK1 inhibitor is comprised within a second nanoparticle. These first and second nanoparticles can be configured as described in any of the above embodiments.

The first and second pharmaceutical compositions of the kit can be used for sequential administration or co-administration, or can be combined to form a further pharmaceutical composition (for subsequent administration); see below for further details on administration approaches.

### Methods of Manufacture

Any polymer-based nanoparticle can be manufactured by providing one or more biodegradable and biocompatible polymers, any of which is optionally (fully or partially) covalently linked to an active compound or a functionalisation group, optionally providing a free (i.e. not bound to polymer) active compound and/or functionalisation group, and deploying an emulsion-solvent evaporation process on the mixture of provided components. Optionally, the resultant nanoparticle (or ‘polymer core’) is subject to thin-film hydration, optionally in the presence of a further active compound, such that the (polymer-based) nanoparticle (or ‘polymer core’) is surrounded by a liposome shell (that optionally contains said further active compound within the lipid bilayer).

### Clinical Applications

Provided is a taxane for use in a method of treating cancer, wherein the taxane is administered simultaneously, separately or sequentially with an SK1 inhibitor. Also provided therefore is a product containing a taxane and an SK1 inhibitor for simultaneous, separate or sequential use in treating cancer.

A range of administration routes is possible, though intravenous administration is preferred.

By ‘treating cancer’ we mean e.g. attempting to and/or achieving any clinically relevant measure of cancer treatment, such as reduction or elimination of cancer (tumour) growth (based on e.g. volume and/or mass), cancer shrinkage (based on e.g. volume and/or mass), and/or improvements in terms of overall survival (OS), progression-free survival (PFS), time to progression (TTP), time to treatment failure (TTF), event-free survival (EFS), time to next treatment (TTNT), objective response rate (ORR) and/or duration of response (DoR).

In preferred embodiments, the taxane is administered after the SK1 inhibitor has been administered, or at least the start of administration of the taxane occurs after the start of administration of the SK1 inhibitor. Where repeated doses are to be administered, this means that, for example, in the first dose (and potentially in subsequent doses), the SK1 inhibitor is administered and then the taxane is administered. Alternatively, the SK1 inhibitor is administered and then two or more doses of taxane are sequentially administered. In such embodiments, the taxane and the SK1 inhibitor can be administered as free agents (e.g. where the agents/actives are not comprised within a nanoparticle). Otherwise, preferably, administration is of a taxane comprised within a first nanoparticle and an SK1 inhibitor comprised within a second nanoparticle (preferably as per any of the above described embodiments). Preferably, the actives (free or comprised within a nanoparticle) are comprised within (respective) pharmaceutical compositions, such as those provided by the kit of the invention.

In alternative, particularly preferred embodiments, the taxane is administered simultaneously with the SK1 inhibitor. Preferably, in such embodiments, this is by administration of a pharmaceutical composition comprising the taxane and the SK1 inhibitor (as described above). In such a composition, the taxane and the SK1 inhibitor can be present as free agents, or both can comprised within a (combined) nanoparticle (as described above), or the taxane can be comprised within a first nanoparticle and the SK1 inhibitor can be comprised within a second nanoparticle (as described above). In the first and third of those options, the pharmaceutical composition could be formed by combining the compositions present in the kit of the invention. Alternatively, in such embodiments, this is by co-administration of a first pharmaceutical composition comprising the taxane and a second pharmaceutical composition comprising the SK1 inhibitor (e.g. the compositions present in the kit of the invention). Co-administration can mean e.g. administration of each active (or composition comprising the same) within no more than 24 hr of each other, preferably no more than 12 hr of each other, preferably no more than 6 hr of each other, preferably no more than 2 hr of each other, preferably no more than 1 hr of each other, preferably no more than 30 min of each other, preferably no more than 10 min of each other, and most preferably no more than 1 min of each other. Administration outside of these time limits can be considered to be sequential administration.

Preferably, the cancer to be treated is one that is responsive to a taxane (preferably docetaxel), particularly a breast, colorectal, lung, ovarian, prostate, liver, renal, gastric (or ‘stomach’), head and neck cancer, or melanoma, particularly breast cancer, lung cancer, head and neck cancer, prostate cancer, or stomach cancer, more particularly breast or prostate cancer. Preferably, the prostate cancer is castrate-resistant (also known as ‘hormone resistant’ or ‘hormone refractory’), i.e. no longer sensitive to hormone (anti-androgen) treatment. Alternatively, or in addition, the prostrate cancer may be local, locally advanced or metastatic.

Within a single dose of the combination therapy, the ratio of taxane to SK1 inhibitor on a weight basis is preferably 1000:1 to 1:1000, more preferably 500:1 to 1:500, more preferably 200:1 to 1:200, more preferably 100:1 to 1:100, more preferably 10:1 to 1:10, most preferably approximately 1:1. For each of the taxane and the SK1 inhibitor, the dosage is preferably 1 mg/kg to 50 mg/kg, preferably 1 or 2 mg/kg to 20 mg/kg, preferably 1 or 2 mg/kg to 10 mg/kg, most preferably approximately 5 mg/kg.

Preferably, particularly when the taxane and the SK1 inhibitor are comprised within a nanoparticle (and more particularly when the taxane is encapsulated by and/or covalently linked to a biodegradable and biocompatible polymer within the nanoparticle, said polymer is surrounded by a liposome shell, and the SK1 inhibitor is contained within the lipid bilayer of the liposome), said method does not induce lymphopenia and/or leukopenia.

### Methods of Treatment

Also provided are methods of treatment corresponding to all of the compound/composition “for use” aspects of the invention detailed above. So, for example, provided is a method of treating cancer in a human or animal (preferably mammalian) individual, said method comprising administering to said individual a taxane simultaneously, separately or sequentially with an SK1 inhibitor.

### General

Please note that wherever the term ‘comprising’ is used herein we also contemplate options wherein the terms ‘consisting of’ or ‘consisting essentially of’ are used instead.

### Examples

### Example 1—Studies in Relation to Free Active Agents

### Methods and Materials

a) Animal Study

Animal study was performed as previously described (Pchejetski, D. et al. Sphingosine kinase-1 as a chemotherapy sensor in prostate adenocarcinoma cell and mouse models. Cancer research 65, 11667-11675, doi:65/24/11667 [pii] (2005) and Pchejetski, D. et al. FTY720 (fingolimod) sensitizes prostate cancer cells to radiotherapy by inhibition of sphingosine kinase-1. Cancer Res 70, 8651-8661, doi:0008-5472.CAN-10-1388 [pii]). Briefly, subcutaneous human prostate and breast cancer xenografts were established in Balb/c nude mice by subcutaneous injection of 1*10⁶ PC-3 metastatic prostate cancer cell and MDA-MB-231 metastatic triple negative breast cancer cell. Three weeks after implantation, mice with prostate tumour were randomized into treatment groups (n=7/group) and treated twice a week for two weeks with: i.v. tail vein injections of: saline, 5 mg/kg docetaxel, 20 mg/kg docetaxel, 5 mg/kg SK1 inhibitor (SK1-II), 5 mg/kg SK1 inhibitor(SK1-II)+5 mg/kg docetaxel. Mice with breast tumour were also randomized into treatment groups (n=7/group) and treated twice a week for two weeks with: i.v. tail vein injections of: saline, 5 mg/kg docetaxel, 5 mg/kg FTY720, and 5 mg/kg FTY720+5 mg/kg docetaxel. One day after the last treatment, all mice were euthanized. Mice and tumours were weighed.

b) Statistical Analysis

Data are presented as the mean values of at least three independent experiments normalised to control±standard error of the mean (SE) calculated using OriginPro. Statistical significance between two groups was conducted by unpaired Student's t test. p value of <0.05 is considered statistically significant.

### Results and Conclusions

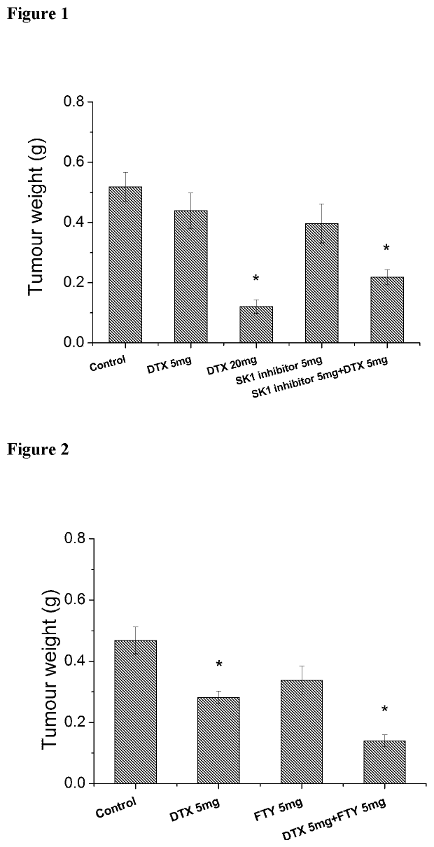

To investigate the synergistic effect of docetaxel and SK1 inhibitor we used animal models of both human prostate tumours and breast tumours. In the control groups, tumours rapidly grew reaching 0.52±0.05 g and 0.47±0.04 g in mice with prostate tumours and breast tumours, respectively, at week five, while combination of 5 mg/kg docetaxel and 5 mg/kg SK1 inhibitor have significantly reduced the tumour weight to 0.22±0.02 g and 0.14±0.02 g respectively (FIG. 1 and FIG. 2). In contrast to the drug combination treatment, individual drug treatments with docetaxel (5 mg/kg) or SK1 inhibitor (5 mg/kg) did not reduce the tumour weight significantly.

### Example 2—Studies in Relation to CSLPHNPs

### Methods and Materials

a) Synthesis and Characterisation of CSLPHNPs.

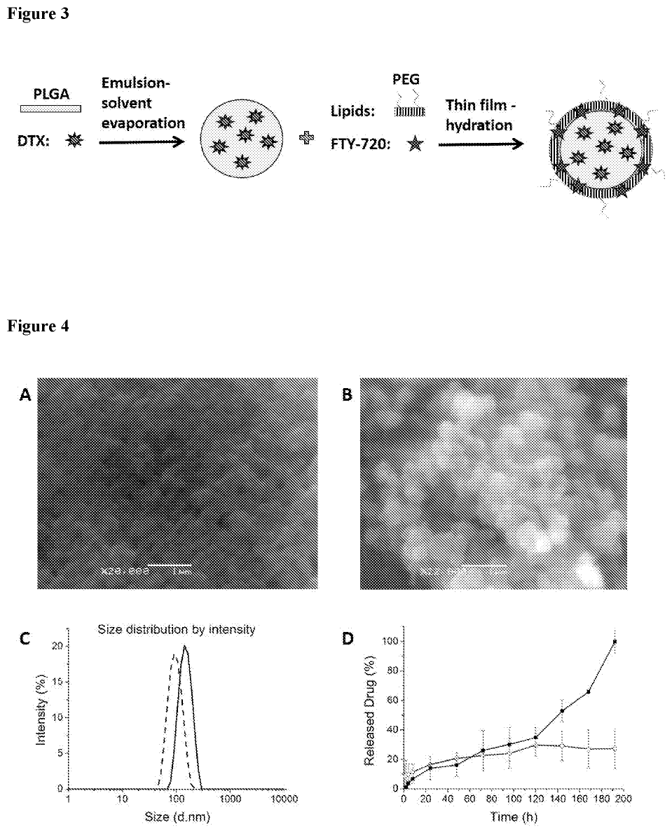

PLGA nanoparticle (NP) cores were prepared by emulsion-solvent evaporation technique (Sengupta, S. et al. Temporal targeting of tumour cells and neovasculature with a nanoscale delivery system. Nature 436, 568-572, doi:10.1038/nature03794 (2005)) (see FIG. 3). Briefly, PLGA and docetaxel 10:1 wt % was dissolved completely in acetone. The entire solution was emulsified into 2% aqueous solution of 80% poly vinyl alcohol (PVA) by slow injection with homogenisation. This mini-emulsion was then added to a 0.2% PVA solution with rapid mixing overnight to evaporate any residual acetone. Nanoparticle-size fraction was recovered by ultrocentrifugation at 10,000, 20,000 and 80,000×g. A mixture of phospholipids, cholesterol and FTY720 were dissolved in chloroform, and then a lipid film was formed in a round bottom flask under reduced pressure using a vacuum rotary evaporator. An aqueous solution of the smallest size fraction of PLGA NPs was added to the film. The resulting suspension was extruded through a 200 nm membrane using a hand held extruder (Avanti, Alabaster) to create the lipid vesicles. The resulting hybrid lipid-polymer nanoparticles were collected by Amicon centrifugal filters (MWCO 100K Da) and washed twice with distilled water.

b) Scanning Electron Microscopy

The surface morphology of the nanoparticles was studied using SEM (JEOL Ltd, UK). The CSLPHNPs were dried on an aluminum stub, coated with gold to obtain a uniform layer of particle and dried overnight. For DLS measurement, the size distribution and zeta potential of the nanoparticles was estimated using a Zetasizer Nano ZS90 (Malvern Instruments, Malvern, UK) with 90° optics and a He—Ne Laser.

c) Liquid Chromatography-Tandem Mass Spectrometry Analysis

To study the release profile of CSLPHNPs in vitro, nanoparticles were suspended in 1 mL of pH5/pH7.4 buffer and were placed in an Amicon dialysis device (MWCO 1000 Da) suspended in 14 mL pH5/pH7.4 buffer. After stipulated time points 2 mL aliquots of water were extracted and replaced with fresh water (pH5.5). The dialysis device was incubated at 37° C. with gentle shaking. Aliquots were extracted from the incubation medium at predetermined intervals, and released drug was quantified by an Agilent6400 Triple Quadrupole LC/MS/MS system. Samples were applied to a Ascentis Express C18 reverse phase column (50×2.1 mm, 2.7 μm) and eluted using an 80:20 acetonitrile:isopropanol (ACN:IPA), water, formic acid gradient (flow rate 0.4 mL/min).

d) Cell Lines and Cell Culture

Androgen insensitive prostate cancer cell lines (PC-3 and DU145) were obtained from DSMZ (Braunschweig, Germany). Cells were maintained in tissue culture flasks or plastic dishes in a humidified atmosphere of 5% CO₂ at 37° C. using Roswell Park Memorial Institute (RPMI) 1640 supplemented with 10% heat-inactivated fetal bovine serum (FBS) (Sigma-Aldrich, UK), 50 U/ml penicillin, 50 μg/ml streptomycin and 2 mM glutamine (Sigma-Aldrich, UK). Cell lines were routinely verified by morphology and growth curve analysis. All experiments were conducted in the absence of serum. Cells were seeded to be 80% confluent by the end of treatment and were treated as indicated in figures' legends. Cell lines were kept in culture for up to 30 passages.

e) Cell Viability

Cells were grown in 96-well plates, starved, and exposed to different treatments as indicated in figure legends. Cellular viability was measured using the 3-(4,5-dimethylthiazol-2-yl)-2,5-diphenyl tetrazolium bromide (MTT; 5 mg/ml) colorimetric assay as already described (Alshaker, H. et al. Leptin induces upregulation of sphingosine kinase 1 in oestrogen receptor-negative breast cancer via Src family kinase-mediated, j anus kinase 2-independent pathway. Breast Cancer Res 16, 426, doi:10.1186/s13058-014-0426-6 (2014)).

f) In Vitro Imaging of Tumour Cells

PC-3 cells were seeded onto 6 well plates and treated with CSLPHNPs loaded with Rhodamine B for the times shown in FIG. 3. After incubation, the cells were washed three times with PBS and fluorescent imaging was performed using fluorescent microscope (Carl Zeiss, USA). The fluorescent intensities in cells were quantitatively analysed by ImageJ.

g) Sphingosine Kinase 1 Activity Assay

SK1 assay was performed using radiolabelling as previously described (Alshaker et al (2014), above, plus: Alshaker, H. et al. Sphingosine kinase 1 contributes to leptin-induced STAT3 phosphorylation through IL-6/gp130 transactivation in oestrogen receptor-negative breast cancer. Breast Cancer Res Treat 149, 59-67, doi:10.1007/s10549-014-3228-8 (2015); and Pchejetski, D. et al. The involvement of sphingosine kinase 1 in LPS-induced Toll-like receptor 4-mediated accumulation of HIF-1alpha protein, activation of ASK1 and production of the pro-inflammatory cytokine IL-6. Immunol Cell Biol 89, 268-274, doi:10.1038/icb.2010.91 (2011)), in conditions favouring SK1 activity and inhibiting SK2 activity.

h) Animal Study

Animal study was performed as previously described (Pchejetski, D. et al. Sphingosine kinase-1 as a chemotherapy sensor in prostate adenocarcinoma cell and mouse models. Cancer research 65, 11667-11675, doi:65/24/11667 [pii] (2005) and Pchejetski, D. et al. FTY720 (fingolimod) sensitizes prostate cancer cells to radiotherapy by inhibition of sphingosine kinase-1. Cancer Res 70, 8651-8661, doi:0008-5472.CAN-10-1388 [pii]). Briefly, subcutaneous human prostate cancer xenografts were established in NSG mice male mice by subcutaneous injection of 1*10⁶ PC-3 cells. Three weeks after implantation, mice were randomized into treatment groups (n=7/group) and treated twice a week for two weeks with: i.v.tail vein injections of: saline, 5 mg/kg FTY720+5 mg/kg docetaxel, empty CSLPHNPs and CSLPHNP (5 mg/kg FTY720+5 mg/kg docetaxel). One day after the last treatment, all mice were euthanized and blood collected for WCC measurement. Mice, tumours and individual organs were weighed and primary tumours were then processed for analysis of SK1 activity (radiolabeling) as described above. Primary tumours, livers, spleens and kidneys were fixed with 4% paraformaldehyde and fluorescent images were obtained using a stereo microscope and quantified using ImageJ.

i) Statistical Analysis

Data are presented as the mean values of at least three independent experiments normalised to control±standard error of the mean (SE) calculated using OriginPro. Statistical significance between two groups was conducted by unpaired Student's t test. p value of <0.05 is considered statistically significant.

### Results

a) Characterisation of CSLPHNPs

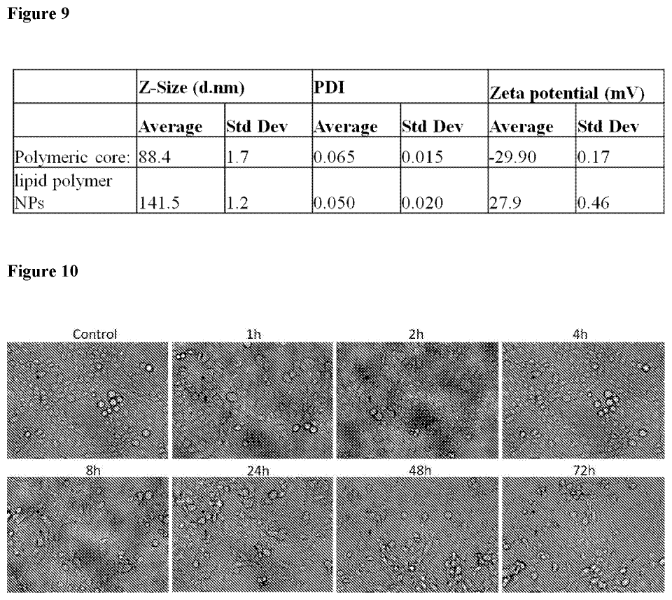

The morphology of CSLPHNPs was characterised by scanning electron microscopy (SEM) (FIGS. 4A and 4B). The SEM images show that the hybrid nanoparticles were dispersed, with a well-defined spherical core-shell structure. The Dynamic light scattering (DLS) particle sizes, poly-dispersity index (PDI) and zeta potentials of PLGA cores and CSLPHNPs are shown in FIGS. 8 and 9. The CSLPHNPs are larger than the PLGA cores (141.5±1.2 nm and 88.4±1.7 nm respectively) (FIG. 4C & FIG. 9) and represent dimensions that are within an ideal size range (i.e. 20-200 nm). Due to the PLGA terminal carboxyl acid, PLGA NPs showed an average negative surface charge of −29.9 mV in phosphate buffered saline (PBS) (FIG. 9).

Nanoparticle translation into clinic is limited by their stability in body environments. CSLPHNPs had high colloidal stability in various biological media showing no significant change in size and polydipersity index (PDI) (FIG. 8), suggesting that they could be stored with little or no aggregation (FIG. 8C). In pure FBS and 10% human plasma solution CSLPHNPs showed an initial ˜15 nm increase in size and 0.20 increase in PDI, after which they maintained size and stability throughout the 5 days study (FIG. 8), suggesting that plasma protein binding was not a significant modifying factor, possibly due to steric repulsion due to the hydrodynamic diameter of the PEG chain, as well as the stable core-shell structure.

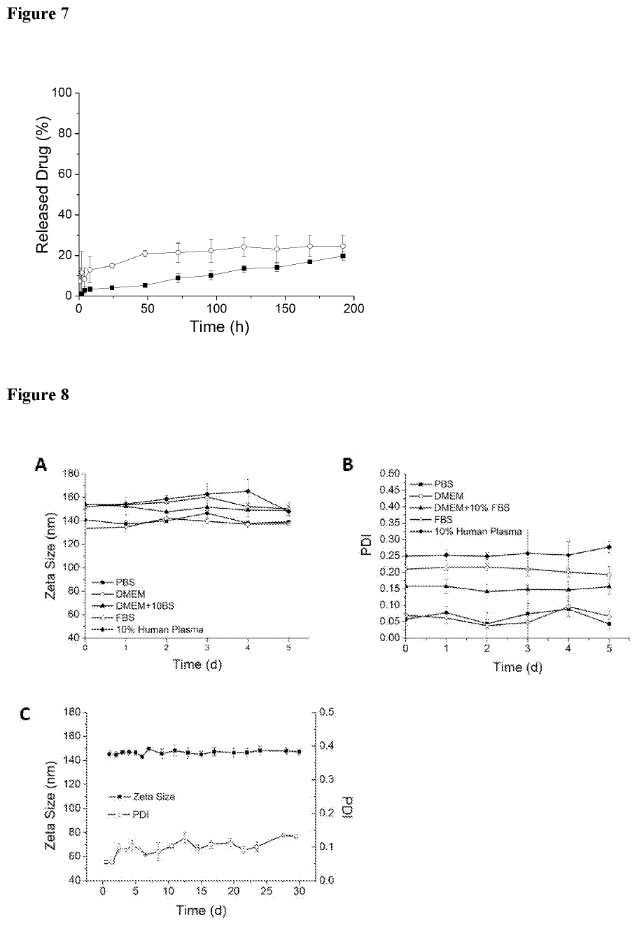

High drug loading and controlled release of contents are two important advantages of CSLPHNPs. The encapsulation of docetaxel and FTY720 in CSLPHNPs was 10% and 70% respectively as confirmed by liquid chromatography-tandem mass spectrometry (LC-MS/MS). The relatively low encapsulation efficiency of docetaxel was probably due to the size selection process of PLGA core synthesis. In vitro release profiles of FTY720 and docetaxel were pH dependent with a “lysosomal” pH5 (FIG. 4D) showing a quicker release than “serum” pH 7.4 (FIG. 7). At pH5, the release pattern of both drugs was initially similar, followed by a surge in FTY720 at 96 h indicating disintegration of the lipid outer layer, reaching 100% at 192 h. Only around 30% of docetaxel was released into the medium, which indicates, that most of the docetaxel is still under protection of the polymeric core. It is, however, possible that in cells the PLGA core may be broken down through enzymatic degradation leading to docetaxel release.

b) In Vitro Studies

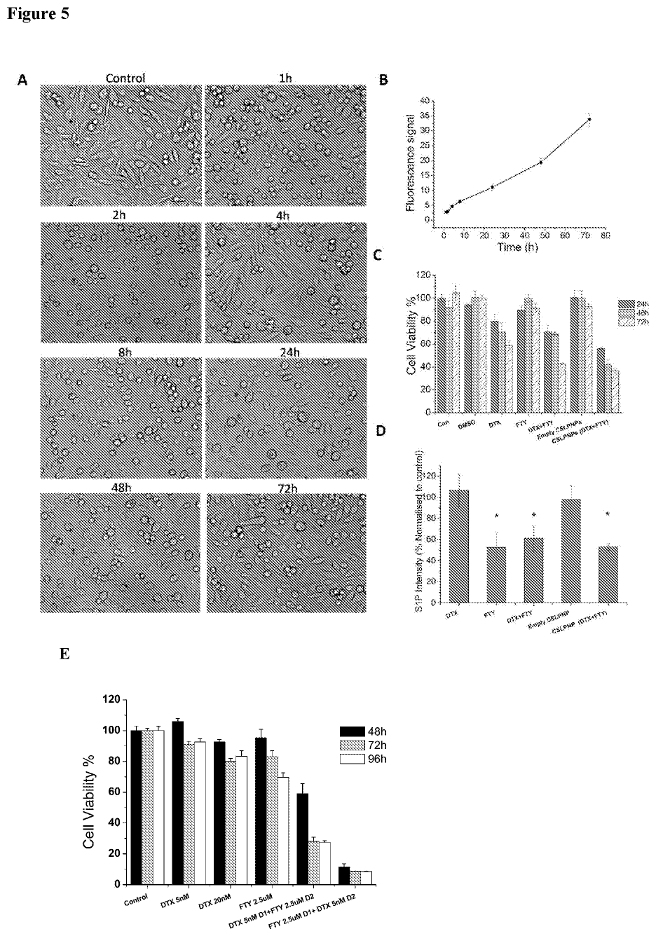

To measure CSLPHNPs cellular uptake, rhodamine B was loaded into the PLGA NPs core instead of docetaxel. Fluorescent microscopy showed clear rhodamine B signal after 24 h exposure, which indicates relatively rapid accumulation of CSLPHNPs within the cells, which was time-dependent (FIGS. 5A,B and 10).

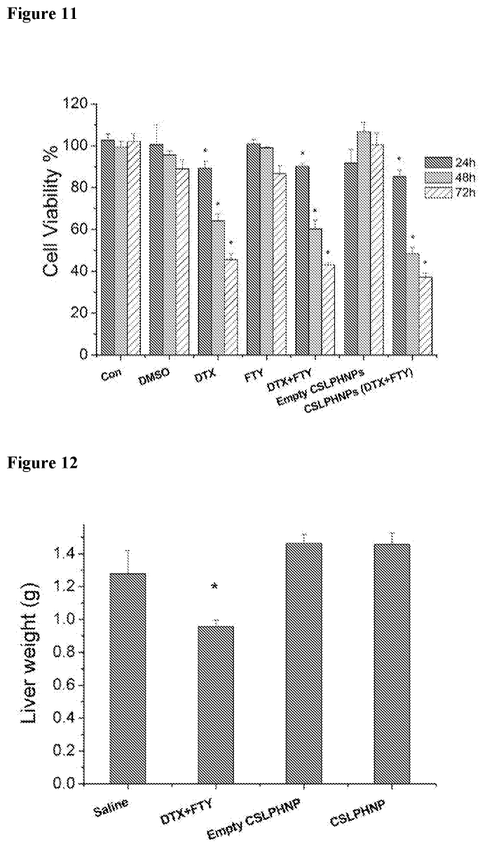

To investigate anticancer properties of CSLPHNPs we used MTT cell viability assay (FIG. 5C). The most effective molar ratio of FTY720:docetaxel in PC-3 and DU145 cells yielded 5 μM:5 nM respectively. Free docetaxel and FTY720 had an additive effect. Empty CSLPHNPs exhibited no significant cytotoxicity in PC-3 cells, while CSLPHNPs containing same amount of FTY720/docetaxel were, 15, 27 and 6% more cytotoxic than free drugs at the 24, 48, and 72 h respectively. Docetaxel has a short circulation half-life of ˜17 hours, FTY720 has a long half-life of 6-9 days and PEG functionalised liposome nanoparticles have half life time>5 days. It is possible that FTY720/docetaxel encapsulation in CSLPHNPs allows stabilisation of their intracellular release (FIG. 4D), providing an increased cytotoxic benefit. In DU145 prostate cancer cells, CSLPHNPs induced greater loss of cell viability compared with free drugs, albeit with more modest effects than compared with those seen with PC-3 cells (FIG. 11).

FIG. 5D shows that all FTY720 containing therapies including CSLPHNPs have significantly reduced SK1 activity in a similar fashion, while docetaxel has induced an insignificant increase. Taken together the data suggest that FTY720 potentiates docetaxel effect through downregulation of SK1, and that the combined nano-formulation improves chemotherapy efficacy possibly through localised release of higher concentration of both drugs inside the cells.

Finally, we noted (and see FIG. 5E) that, when using free drugs in the MTT cell viability assay, a sequence of FTY720 followed by docetaxel was the optimum administration approach.

c) In Vivo Studies

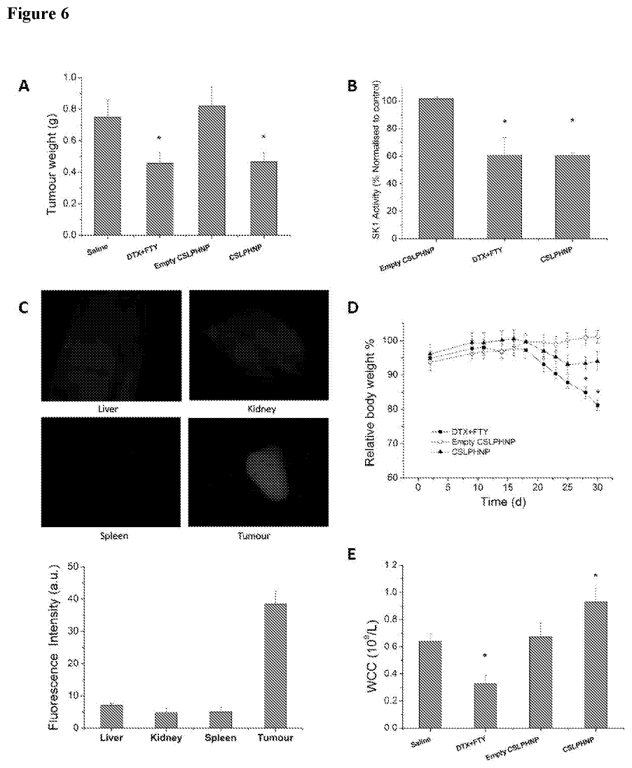

To investigate the in vivo effects of CSLPHNPs we used an animal model of human prostate tumours. NOD scid gamma (NSG) immunodeficient mice were inoculated with PC-3 cells. Tumours were grown for three weeks and then treated for two weeks with saline, free drug combination (5 mg/kg FTY720+5 mg/kg docetaxel), empty CSLPHNPs labelled with CF488 fluorophore or CSLPHNPs (5 mg/kg FTY720+5 mg/kg docetaxel+CF488). In the saline and the empty CSLPHNPs groups, tumours rapidly grew reaching 0.75±0.11 g and 0.82±0.12 g respectively by day 30, while free therapies and CSLPHNP have significantly reduced the tumour weight to 0.46±0.07 g and 0.47±0.06 g respectively (FIG. 6A). Similar to in vitro studies, all treatments containing FTY720 have significantly reduced tumour SK1 activity (FIG. 6B).

CSLPHNPs have excellent tumour targeting capability with ˜40% of the drug dose delivered to the tumour (FIG. 6C), suggesting a high selection towards unorganised tumour vessels. There was a minimal penetration into other tissues (FIG. 6C).

Chemotherapy-induced whole body toxicity is of utmost clinical importance as it is the key limiting factor to administration of effective chemotherapy doses in cancer patients. In NSG mice, systemic FTY720+docetaxel therapy, while having good antitumour efficacy (FIG. 6A) induced a 20% reduction in total body weight (FIG. 6D), significantly reducing liver size (FIG. 12) and general mouse wellbeing. In contrast, CSLPHNPs did not affect body weight (FIG. 6D) or liver weight (FIG. 12) demonstrating the best combination between efficacy (similar to free drugs) and lack of toxicity (similar to chemotherapy-free nanoparticles).

The major obstacle for FTY720 use in cancer patients is significant lymphopenia induced by this drug due to T-cell sequestration to lymph nodes. Haematological assessment showed that free FTY720 has significantly reduced white cell count after 2 weeks of administration (FIG. 6E). In contrast, all CSLPHNPs formulations showed no decrease in white cell count, effectively overcoming FTY720-induced lymphopenia. This is the first study demonstrating a mechanism to overcome FTY720-induced lymphopenia, allowing its potential use in cancer patients. Overall, our data demonstrate a clear advantage of CSLPHNPs over free FTY720 plus docetaxel systemic therapy, specifically by tumour targeting and reducing unwanted side effects.

### Conclusions

CSLPHNPs have high serum stability and a long shelf life. CSLPHNPs showed a steady uptake by tumour cells, sustained intracellular drug release and superior in vitro efficacy to combined free therapies (in turn superior to single free therapies). In a mouse model of human prostate cancer, CSLPHNPs showed excellent tumour targeting and significantly lower side effects compared to free drugs. Importantly CSLPHNPs have completely abrogated lymphopenia induced by FTY720. Our results provide the first preclinical evidence of a therapeutic advantage of nano-formulated combination of FTY720 and docetaxel over free systemic therapy in advanced prostate cancer and provide a mechanism to overcome FTY720-induced lymphopenia, allowing its potential use in cancer patients.

### Example 3—Studies in Relation to Uncoated NPs

### Methods and Materials

a) Drug Modification, Characterization and Conjugation

### Synthesis of Amine Protected FTY720:

FTY720 (1 eq.) was dissolved in dichloromethane (DCM) and incubated with di-tert-butyl dicarbonate (tBoc) (1 eq.) and diisopropylethylamine (DIPEA) (0.5 eq.). Reaction was stopped and the organic layer was dried using anhydrous sodium sulfate. The mixture was purified by column chromatography and characterised by H¹NMR spectroscopy.

### Synthesis of Amine Deprotected Docetaxel:

Docetaxel (1 eq.) was dissolved and incubated in a solution of 33% Trifluoroacetic acid (TFA) in DCM and the reaction was quenched by evaporating DCM and TFA under pressure.

### Conjugation of FTY720 to PLGA:

PLGA (50:50)(1 eq.) dissolved in dimethyl formamide (DMF) dicyclo carbodiimide (DCC) (0.5 eq.), dimethyl amino pyridine (DMAP) (0.5 eq.) and FTY-tBoc (1.1 eq.) were added. The polymer was precipitated using ice-cold diethyl ether.

### Conjugation of Docetaxel and Glucosamine to PLGA:

PLGA (50:50)(1 eq.) dissolved in DMF, N,N,N′,N′-Tetramethyl-O-(1H-benzotriazol-1-yl)uronium hexafluorophosphate (0.5 eq.), DIPEA (0.5 eq.), amine deprotected docetaxel (1.1 eq.) or glucosamine (1.1 eq.) were added. The polymer was precipitated using ice-cold diethyl ether.

### Loading Studies:

Concentration based calibration plots were constructed using UV-visible spectrophotometer (Hewlett Packard) for FTY720 (λ=266 nm), docetaxel (λ=252 nm) and glucosamine (λ=242 nm) in chloroform.

### Nanoformulation:

PLGA-FTY720, PLGA-Docetaxel and PLGA-glucosamine were dissolved in acetone. The entire solution was emulsified into 2% aqueous solution of 80% hydrolyzed poly vinyl alcohol (PVA) by slow injection with constant homogenization and added to 0.2% aqueous solution of PVA with rapid mixing. Nanoparticle-size fraction was recovered by ultrocentrifugation at 80,000 g. Particle size was determined by dynamic light scattering (DLS).

Nanoparticle characterization was done using Scanning Electron Microscope (SEM) (JEOL SEM JSM 804A, UK), Transmission Electron Microscope (TEM) (JEOL 2011, UK) and Zetasizer Nano ZS90 (Malvern Instruments, Malvern, UK).

Nanoparticle Release profile was studied in pH5 phosphate buffered saline using UV-Vis spectrophotometer (Perkin Elmer, UK).

b) Cell Culture

MDAMB-231, 4T1 and PC-3 cancer cell lines were purchased from ATCC (Manassas, Va., USA), maintained in Dulbecco's Modified Eagle's Medium (DMEM) with 10% heat-inactivated foetal calf serum (FCS) (FirstLink, Birmingham, UK), 50 U/ml penicillin, 50 μg/ml streptomycin and 2 mM glutamine (Sigma-Aldrich, St. Louis, Mo.). Cell lines were kept in culture for up to 30 passages. Cells were seeded to reach 70-80% confluence by the end of the treatment.

c) In Vitro Imaging and Uptake of PLGA NP

PC-3 cells were seeded onto 6 well plates and treated with PLGA NPs loaded with Rhodamine B for 48 h. After incubation, the cells were washed three times with PBS and fluorescent imaging was performed using fluorescent microscope (Carl Zeiss, USA). The fluorescent intensities in cells were quantitatively analysed by ImageJ. The temporal uptake of PLGA NP in PC-3 cells were also quantified using flow cytometry at varies time point.

d) MTT Proliferation Assay

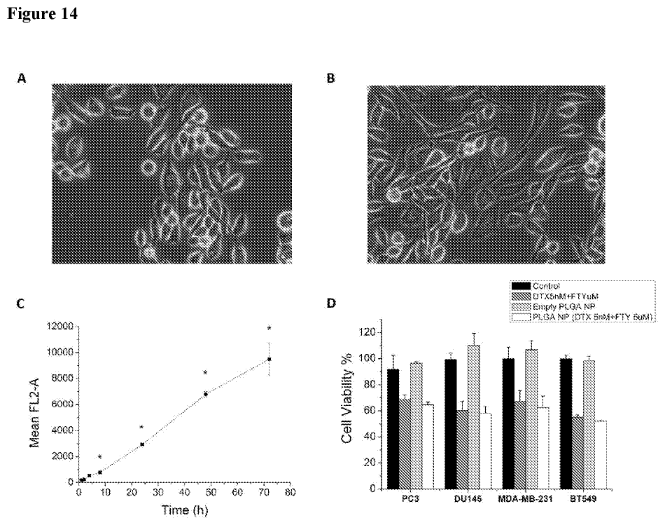

MTT cell viability assay in two prostate (PC3 and DU145) and two breast (MDA-MB-231 and BT549) cancer cell lines. Cells were seeded in 96-well plates and incubated for 24 h, then starved for 24 h and then incubated 48 h with treatments indicated in the FIG. 14 D. MTT 5 mg/ml was added to each well. After 3.5 h of incubation at 37° C., supernatant was aspirated and formazan crystals were dissolved in 0.5 M dimethylformamide and 20% SDS. Optical density was read at 570 nm using microplate reader (Tecan Sunrise™, Maennedorf, Switzerland).

e) Animal Studies

Human breast cancer xenografts were established in Balb/c mice by injection of 1*10⁶ 4T1 cells into their mammary pad. Two weeks after implantation, mice were randomized into treatment groups (n=6/group) and treated twice a week for two weeks with: i.v.tail vein injections of: saline, 3 mg/kg FTY720+5 mg/kg docetaxel, empty PLGA NPs, PLGA NP1 (3 mg/kg FTY720+5 mg/kg docetaxel), PLGA NP2 (2 mg/kg FTY720+2 mg/kg docetaxel). One day after the last treatment, all mice were euthanized and blood collected. Mice, tumours and individual organs were weighed.

f) Data Representation and Statistical Analysis

The statistical significance of differences between the means of two groups was evaluated by unpaired two-sided student's t test, and the overall significance of multiple groups was evaluated by ANOVA. Calculations were performed using Microsoft Excel software. p value of <0.05 is considered statistically significant.

### Results

a) Characterisation of NPs

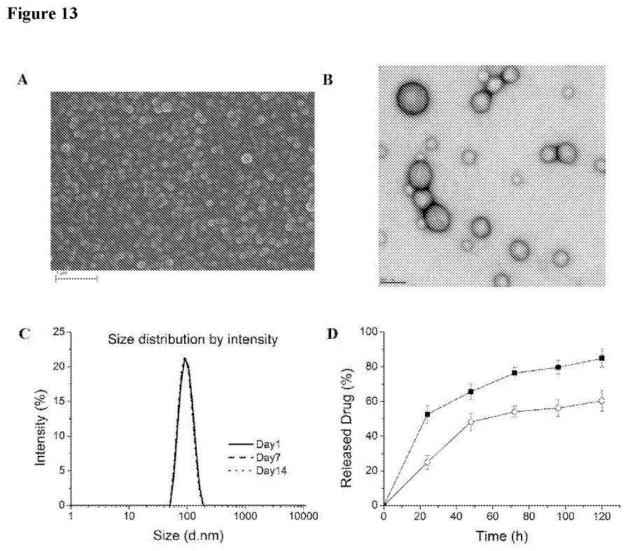

PLGA NPs size distribution was evaluated using scanning electron microscopy (FIG. 13A), transmission electron microscopy (FIG. 13B) and dynamic light scattering (DLS) (FIG. 13C) and confirmed to be between 100-130 nm. PLGA diameter was measured at 91.51±1.37 nm, with polydispersity index 0.05±0.005 and Zeta potential of −14.0±0.6 mV. To assess the drug release profile, PLGA NPs were incubated in release buffer pH5 (replicating lysosomal environment) and drug release was quantified over 120 h by UV-spectrometry (FIG. 13D). As expected, the more labile ester linkage of FTY720 results in increased release of the drug over time in comparison to the more stable docetaxel amide bond. At 24 h, the release of FTY720 was approximately double that of docetaxel. FTY720 release continued until plateau at about 80% after 72 hrs.

b) In Vitro Studies

Antitumour efficacy of PLGA NPs was evaluated using MTT cell viability assay in PC-3, DU145, MDA-MB-231 and BT549 cells. A significant loss of cell viability was observed in cells treated with PLGA NPs at 48 h (FIG. 14D). PLGA NPs labelling with rhodamine B revealed their internalisation in PC3 prostate cancer cells (FIG. 14B). FACS analysis showed a rapid internalisation already within first 24 hours, and the process is continued throughout to 72 h (FIG. 14C).

c) In Vivo Studies

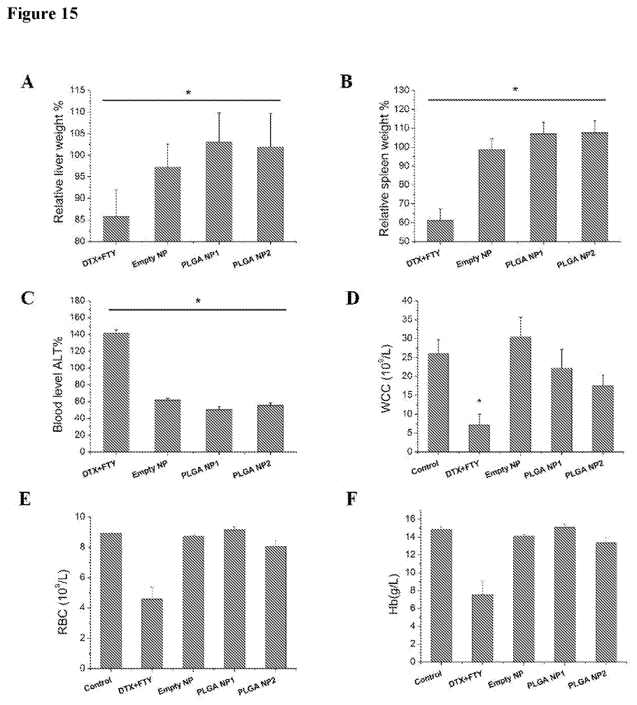

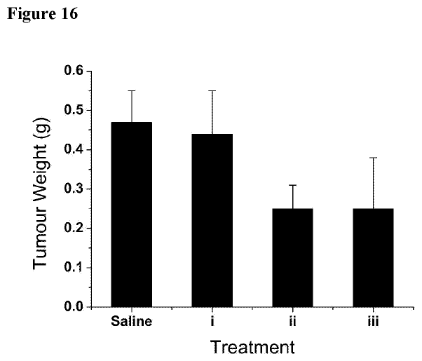

In vivo efficacy of PLGA NPs was assessed in BALB/c mice injected with 4T1 cells into their mammary pad. Mice were randomised into groups of 6 animals receiving intravenous: control (PBS), empty nanoparticle, systemic docetaxel+FTY720 (5 mg/kg+3 mg/kg), PLGA NP1 (docetaxel+FTY720 5 mg/kg+3 mg/kg) and PLGA NP2 (docetaxel+FTY720 2 mg/kg+2 mg/kg). Treatment was started 11 days post tumour implantation and lasted for 14 days. Both PLGA NP1 and PLGA NP2 had a similar efficacy and led to a ˜2.5 fold reduction in tumour volume, which was comparable to free drug combination. To investigate PLGA NPs targeting properties, rhodamine B labelled PLGA NPs were injected in the last treatment cycle. Epifluorescence analysis of tumours and organs showed an almost specific targeting to tumour sites. This targeting has led to a 3- and 2-fold reduction of lymphopenia and anemia, respectively, induced by free treatment (FIG. 15D-F). PLGA NPs targeting improved the median survival rate of free drugs, and reduced cytotoxic effects of free therapy such as body weight loss, organ weight loss (FIG. 15B) and liver toxicity (FIGS. 15A and 15C).

### Conclusions

The results indicate that FTY720 acts in synergy with docetaxel, potentially via a mechanism that involves FTY720-mediated SK1 inhibition, with the PLGA NP platform possibly facilitating this by the sequential release of FTY720 followed by docetaxel. Most importantly, our study provides first evidence that PLGA NP encapsulation of FTY720 can reduce systemic lymphopenia and anemia, making it a candidate drug for use in cancer patients. We has also showed that PLGA NP encapsulation provides reduced chemotherapy toxicity and FTY720/docetaxel combination may have a potential therapeutic use in clinical cancer treatment.

### Example 4—Studies in Relation to Uncoated, Singular NPs

Materials and methods were essentially as per Example 3, except that two different species of singular NPs were made in addition. For a first, docetaxel NP, the methodology above was followed except that PLGA-FTY720 was omitted. For a second, FTY720 NP, the methodology above was followed except that PLGA-Docetaxel was omitted. Animal studies were conducted essentially as per Example 2, except that treatment consisted of saline or (i) FTY720 nanoparticle (5 mg/kg) on day 1, docetaxel nanoparticle (5 mg/kg) on day 2, (ii) Combined docetaxel and FTY720 nanoparticle (5 mg/kg of each active), (iii) Simultaneous administration of FTY720 and docetaxel nanoparticles (mixed, each at 5 mg/kg). Simultaneous administration of the two actives, via singular or combined NPs, proved more effective in terms of decreasing tumour weight compared with sequential treatment (FTY720 NP followed by docetaxel NP).

1. A pharmaceutical composition comprising a taxane and a sphingosine kinase 1 (SK1) inhibitor.

2. The pharmaceutical composition according to claim 1, wherein the taxane is docetaxel and/or the SK1 inhibitor is fingolimod.

3. The pharmaceutical composition according to claim 1, wherein the taxane and the SK1 inhibitor are both comprised within a nanoparticle.

4. The pharmaceutical composition according to claim 3, wherein said nanoparticle is configured such that, in vivo, release of the SK1 inhibitor from the nanoparticle starts at or before release of the taxane from the nanoparticle, and/or initial release of the SK1 inhibitor from the nanoparticle occurs at the same or a greater rate than initial release of the taxane from the nanoparticle.

5. The pharmaceutical composition according to claim 3, wherein each of the taxane and the SK1 inhibitor is encapsulated by and/or covalently linked to a biodegradable and biocompatible polymer within the nanoparticle.

6. The pharmaceutical composition according to claim 5, wherein the SK1 inhibitor is covalently bound to a biodegradable and biocompatible polymer via an ester linkage, and wherein the taxane is bound to a biodegradable and biocompatible polymer via an amide linkage.

7. The pharmaceutical composition according to claim 3, wherein the taxane is encapsulated by and/or covalently linked to a biodegradable and biocompatible polymer within the nanoparticle, said polymer is surrounded by a liposome shell, and the SK1 inhibitor is contained within the lipid bilayer of the liposome.

8. The pharmaceutical composition according to claim 1, wherein the taxane is comprised within a first nanoparticle and the SK1 inhibitor is comprised within a second nanoparticle.

9. The pharmaceutical composition according to claim 8, wherein said first and second nanoparticles are configured such that, in vivo, release of the SK1 inhibitor from the second nanoparticle starts at or before release of the taxane from the first nanoparticle, and/or initial release of the SK1 inhibitor from the second nanoparticle occurs at the same or a greater rate than initial release of the taxane from the first nanoparticle.

10. The pharmaceutical composition according to claim 8, wherein each of the taxane and the SK1 inhibitor is encapsulated by and/or covalently linked to a biodegradable and biocompatible polymer within the first and second nanoparticles, respectively.

11. The pharmaceutical composition according to claim 10, wherein the SK1 inhibitor is covalently bound to a biodegradable and biocompatible polymer via an ester linkage and wherein the taxane is bound to a biodegradable and biocompatible polymer via an amide linkage.

12. A kit comprising a first pharmaceutical composition comprising a taxane and a second pharmaceutical composition comprising an SK1 inhibitor.

13. The kit according to claim 12, where, in the first pharmaceutical composition, the taxane is comprised within a first nanoparticle and, in the second pharmaceutical composition, the SK1 inhibitor is comprised within a second nanoparticle.

14. The kit according to claim 13, wherein said first and second nanoparticles are configured such that, in vivo, release of the SK1 inhibitor from the second nanoparticle starts at or before release of the taxane from the first nanoparticle, and/or initial release of the SK1 inhibitor from the second nanoparticle occurs at the same or a greater rate than initial release of the taxane from the first nanoparticle.

15. A method of treating cancer, comprising administering a taxane simultaneously, separately or sequentially with an SK1 inhibitor.

16. The method according to claim 15, wherein the taxane is administered after the SK1 inhibitor has been administered.

17. The method according to claim 15, wherein the taxane is administered simultaneously with the SK1 inhibitor, by administration of a pharmaceutical composition comprising the taxane and the SK1 inhibitor, by co-administration of a first pharmaceutical composition comprising the taxane and a second pharmaceutical composition comprising the SK1 inhibitor, or by administration of a pharmaceutical composition formed by the combination of a first pharmaceutical composition comprising the taxane and a second pharmaceutical composition comprising the SK1 inhibitor.

18. The method of claim 15, wherein said cancer is breast cancer or prostate cancer.

19. The method according to claim 18, wherein said prostate cancer is castrate-resistant.
